(19) United States
(12) Patent Application Publication
(10) Pub. No.: US 2008/0009695 A1
(43) Pub. Date: Jan. 10, 2008

(54) MULTI-MODALITY AUTOMATIC EXAM START NOTIFICATION AND PATIENT INFORMATION AUTO-QUERY MECHANISM

(76) Inventors: Claudio Patricio Mejia, Wauwatosa, WI (US); Scott Raymond Kosloske, Slinger, WI (US); Eric St. Pierre, Brookfield, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/552,330

(22) Filed: Oct. 24, 2006

Related U.S. Application Data
(60) Provisional application No. 60/800,481, filed on May 15, 2006.

(51) Int. Cl. A61B 5/05

(52) U.S. Cl. 600/407

(57) ABSTRACT

A system and method for beginning an exam procedure in multiple modality systems during the performance of a physiology procedure. Upon the initiation of a physiology procedure in a physiology workstation, the physiology workstation automatically generates an exam notification message to each modality system required during the performance of the physiology procedure. Upon receiving the start exam notification message, each modality system automatically generates a patient information query back to the physiology workstation requesting patient information and exam information. The physiology workstation returns identical patient information and exam information to each of the modality systems, thereby ensuring that each of the modality systems is operating utilizing the same patient information and exam information. The generation of both the start exam notification message and the patient information query are automatically generated, such that an exam procedure can be initiated by a single manual entry into the physiology workstation, thus eliminating the requirement of manual initiation of each of the individual modality systems.

### CROSS-REFERENCE TO RELATED APPLICATION

The present application is based on and claims priority to U.S. Provisional Patent Application Ser. No. 60/800,481, filed on May 15, 2006.

### FIELD OF THE INVENTION

The subject matter described herein generally relates to a system and method for integrating the operation of the various modality systems required to perform a hemodynamic or electrophysiology procedure, such as in a catheter lab. More specifically, the subject matter described herein relates to a method and system for integrating an automatic exam start notification message to multiple modalities to reduce the steps necessary to begin a physiology procedure, such as in a cardiac catheterization laboratory (cathlab).

### BACKGROUND OF THE INVENTION

The present invention generally relates to a system and method for integrating the operation of the various modality systems required to perform a hemodynamic or electrophysiology procedure, such as in a catheter lab. More specifically, the present invention relates to a method and system for integrating an automatic exam start notification message to multiple modalities to reduce the steps necessary to begin a physiology procedure, such as in a cardiac catheterization laboratory (cathlab).

Today, physiology workstations are used in cathlabs, hemodynamic (HD) labs and electrophysiology (EP) labs to conduct various tests and procedures. The physiology workstation receives and stores information relating to the catheter procedure being performed in the lab, including information obtained from different pieces of equipment that may belong to different modalities. When a hemodynamic or electrophysiology procedure is performed, the first step in the exam/study is to start the procedure within the physiology workstation. In many hemodynamic or electrophysiology procedures, the exam/study needs to be started in both the physiology workstation as well as in the different modality systems required for the performance of the procedure. As an example, in some hemodynamic procedures, an x-ray angiography system is required for performance of the procedure and the exam in the x-ray system must be manually initiated by an operator.

In current systems, the operator must initially manually begin the sequence of events needed to perform the study by first starting the study within the physiological workstation. Once the study has begun within the physiological workstation, the operator must start the exam/study in each modality, such as the x-ray system. Once the procedure has been started on the modality system (x-ray system), the modality system communicates with the physiology workstation such that patient and exam information corresponding to the individual undergoing the procedure is exchanged between the physiology workstation and the modality system. Once the patient information has been exchanged, both the modality system and the physiology workstation contain the same patient and exam information and the procedure can begin.

Similarly, for an EP procedure, the study/exam needs to first be started within the physiology workstation, as in a hemodynamic procedure. Once the study has been initiated within the physiology workstation, depending upon the type of procedure performed, an exam/study also needs to be started in one or more modality systems. As an example, in some electrophysiology procedures, the exam/study needs to be started in both an ultrasound and x-ray modality system.

During a study that includes multiple modality systems, if the sequence of events is not properly performed by the operator, unnecessary delays are introduced into starting the procedure in the various pieces of equipment, resulting in a possible discontinuity in the information flow between the systems. Further, the requirement that the operator manually begin the study/exam in each of the modality systems, as well as the physiology workstation, introduces unwanted delays in the starting of the procedure.

### BRIEF DESCRIPTION OF THE INVENTION

A system and method that eliminates the need to perform the manual steps of initiating a physiology procedure in each piece of equipment that is involved in performing the procedure, thereby reducing the number of steps and time required to begin the physiology procedure is provided.

An embodiment of a combined system required to carry out a physiology procedure on a patient includes a physiology workstation and one or more modality systems that perform various functions/operations on the patient, such as an x-ray modality system or an ultrasound modality system. During the performance of a physiology procedure, each of the modality systems performs a function and returns patient monitoring information to the physiology workstation for storage, review and display. Since many physiology procedures require multiple modality systems to perform a procedure, each modality system must be initiated prior to beginning the procedure.

Upon initiation of the test procedure at the physiology workstation, the physiology workstation identifies which modality systems are required for the performance of the physiology procedure. Once the required modalities are identified, the physiology workstation generates a start exam notification message to each modality system required for the procedure. The start exam notification message is automatically sent from the physiology workstation to each of the modalities upon the manual initiation of the procedure at the workstation.

Each of the modality systems are configured such that upon receipt of the start examination notification message, the modality system automatically generates a patient information query back to the physiology workstation requesting patient identification information and exam information. The automatic query from the modality system back to the physiology workstation ensures that each of the modality systems receives the same patient identification information such that the patient identification information within the physiology workstation is synchronized to the patient identification information within each of the modality systems. Alternatively, the request for patient information could be directed to a departmental scheduling system or an ADT system.

In addition to initiating the physiology procedure at the physiology workstation, it is also possible to initiate the procedure at any one of the modality systems in communication with the physiology workstation. When the procedure is initiated at one of the modality systems, the modality system signals the beginning of the procedure to the physiological workstation. Since the physiological workstation is typically in communication with a data storage location, such as the hospital information system, the physiology workstation can retrieve patient identification information and exam information for the patient undergoing treatment. Upon retrieving this data, the physiology workstation generates exam notification messages to each of the modality systems that are required for the procedure being performed. Thus, the operator can begin a procedure by manually indicating the desire to begin the procedure at only one of the modality systems.

### BRIEF DESCRIPTION OF THE DRAWINGS

The drawings illustrate the best mode presently contemplated of carrying out the invention. In the drawings:

FIG. 1 is a schematic representation of the communication between the physiology workstation and one or more modality systems used during the performance of a hemodynamic or electrophysiology procedure;

FIG. 2 is an alternate configuration of the interaction between the physiology workstation and the plurality of modality systems during the performance of a hemodynamic or electrophysiology procedure; and

FIG. 3 is a flowchart illustrating the operational steps performed upon the initiation of a procedure either in the physiology workstation or one of the modality systems.

### DETAILED DESCRIPTION OF THE INVENTION

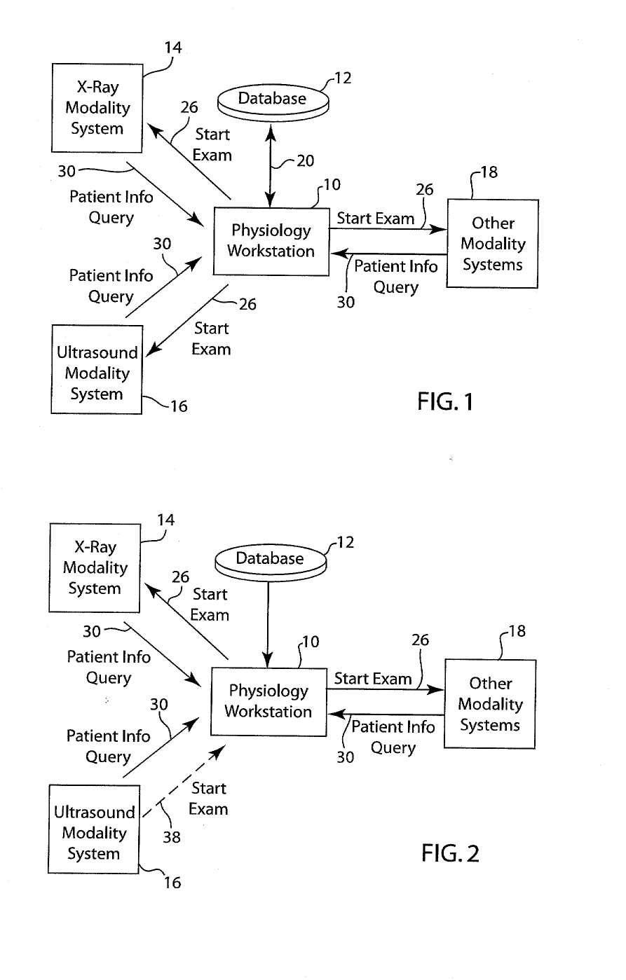

FIG. 1 illustrates a physiology workstation 10 that can be located in the control room or procedural room of a catheter lab, hemodynamic (HD) lab or electrophysiology (EP) lab and can be utilized in connection with HD, EP or ablation procedures, among other things. The physiology workstation 10 may integrate, among other things, real-time hemodynamic information, real-time intracardiac echography, fluoroscopic images, mapping data and pre-surgery planning CT and MR images. The physiology workstation 10 offers integrated monitoring and review of hemodynamic, EP, patient and mapping information, as well as stored and real-time diagnostic images, ECG signals and IC signals.

The physiology workstation 10 is operable to acquire patient monitoring data, whether it be hemodynamic or electrophysiological data, from the patient, such as during the performance of a catheterization procedure. In a networked system, the physiology workstation 10 communicates the received monitoring data to a central data storage device or server 12 that stores the patient monitoring data for access and review by a physician from a remote review station. In addition to storing monitoring data onto the server 12, the physiology workstation 10 can also retrieve patient information from the server 12 that may be required during the completion of a physiology procedure.

As an example, the physiology workstation 10 can receive signals from various different devices, such as intracardiac signals from EP catheters, patient monitoring signal (e.g. from a blood pressure cuff, SpO2 monitor, temperature monitor, CO₂ level, and the like), ECG signals from surface ECG leads, pressure signals from open lumen catheters and intracardiac signals. Further, the physiology workstation 10 can also receive other information from modality systems that are required to conduct the physiology procedure on the patient. In the embodiment of the invention illustrated in FIG. 1, the physiology workstation 10 can receive imaging data from an x-ray modality system 14, ultrasound images from an ultrasound modality system 16, or other types of images/data from various different modality systems, as generally shown by reference numeral 18. As an example, the other modality systems could include x-ray angiography systems, fluoroscopic imaging data from a fluoroscopic modality system or any other type of modality system that may be required for the performance of a physiology procedure.

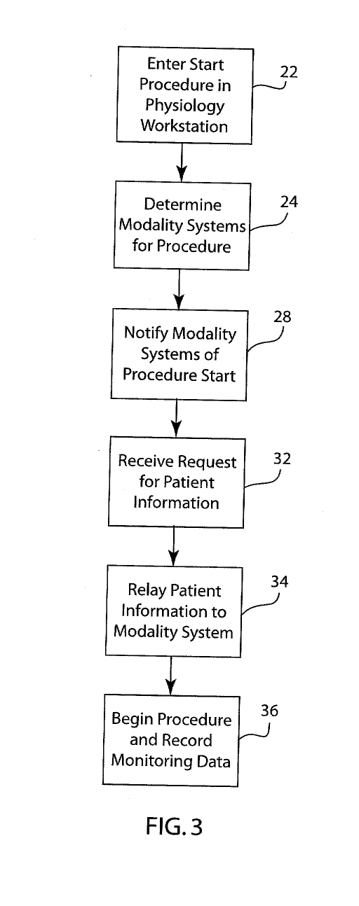

When a clinician/physician desires to begin a physiology procedure, whether the procedure is scheduled or is being performed on an emergency basis, the physician initially begins the procedure by identifying the patient in the physiology workstation 10. The identification of the patient typically requires the physician/clinician to utilize the physiology workstation 10 to access patient information/records from the server 12 using a network connection 20. If the patient information is not contained within the server 12, the operator can manually enter the required information into the physiology workstation 10 through a manual entry means, such as a keyboard. In addition to retrieving patient information from the server 12, the operator also selects the type of physiology procedure that is going to be performed on the patient, as is illustrated in steps 22 and 24 of FIG. 3. As an example, if the operator is going to conduct a hemodynamic catheter procedure, the procedure may require various different operating components in addition to the x-ray modality system 14 shown in FIG. 2. As illustrated in FIG. 1, the physiology workstation 10 is in data communication with the x-ray modality system 14, the ultrasound modality system 16 and various other modality systems 18. Thus, the physiology workstation 10 can communicate with the various modality systems 14, 16 and 18 during the performance of a physiology procedure on a patient.

As illustrated in FIGS. 1 and 3, once the operator has indicated the start of a physiology procedure in the physiology workstation 10, the physiology workstation 10 initially identifies which modality systems are required for the physiology procedure, as indicated in step 24. Once the physiology workstation 10 has determined which modality systems are required, the physiology workstation 10 relays a “start exam” notification 26 to each of the modality systems 14, 16 and 18 required for the procedure, as illustrated by step 28 in FIG. 3. Although FIG. 1 illustrates the notification message 26 being sent to each of the modality systems 14, 16 and 18, it should be understood that the “start exam:” notification message 26 will preferably be sent to only the modality system or systems that are required for performance of the physiology procedure to be carried out on the patient. As an example, if the hemodynamic procedure requires the use of the x-ray modality system 14 but not the operation of the ultrasound modality system 16, the physiology workstation 10 will send the “start exam” notification 26 only to the x-ray modality system 14.

Once the modality system receives the “start exam” notification 26, the modality system 14 must obtain the patient and examination information for the procedure that is being conducted. In the embodiment illustrated in the Figures, each modality system 14 that receives the “start exam” notification 26 begins preparing for the required operation for the identified procedure. Initially, each modality system required for the performance of the physiology procedure automatically generates a patient information query 30 to the physiology workstation 10 requesting patient and exam information corresponding to the individual undergoing the procedure. Each modality system 14, 16 and 18 that is involved with the procedure being performed needs patient information such that the results of the procedure can be properly correlated with the patient undergoing treatment. The patient information may include information such as the patient's name, sex, age, patient identification number and any other information required to correlate the procedural results with the patient undergoing the procedure. In addition to the workstation 10, the patient information could be received from other sources, such as a departmental scheduling system or an ADT system.

Once the physiology workstation 10 receives the request for patient information, as illustrated in step 32 in FIG. 3, the physiology workstation 10 exchanges the patient information with the requesting modality 14, 16 or 18. Once the patient information is relayed to the requesting modality system, as illustrated in step 34, the requesting modality system will be properly synchronized with the physiology workstation 10, including containing the same patient identification information. Thus, all of the modality systems include identical patient and exam information such that the procedural results can be properly correlated and stored within the physiology workstation 10.

Once each of the modality systems that are required for the performance of the physiology procedure have received the patient identification and exam information, the physiology workstation 10 and the modality systems 14, 16 or 18 can begin the performance of the physiology procedure, as illustrated in step 36 of FIG. 3. As can be appreciated in FIGS. 1 and 3, the physiology procedure to be conducted is initiated by a manual entry into the physiology workstation 10. Upon receiving the manual entry, the physiology workstation generates an automatic “start exam” notification message to each of the modality systems that are required for the procedure. Upon receiving the automatically generated start procedure notification message, the modality systems involved in the procedure obtain patient identification information and exam identification information from the physiology workstation 10. Once this information has been received, each of the modality systems can begin to carry out the examination procedure and relay the monitoring data to the physiology workstation 10 for viewing and storage in a conventional manner. The physiology workstation 10 may selectively record the monitoring data at the remote server 12, which may be part of the hospital information system (HIS).

Although the embodiment of the invention shown in FIG. 1 contemplates an automatic notification sent to each of the modality systems involved in the procedure, it should be understood that the automatic “start exam” notification message could be sent to any type of equipment that is required for performing the physiology procedure on the patient such that all of the modality systems and equipment required for performing the procedure are provided with the identical patient and examination information.

Referring now to FIG. 2, thereshown is an alternate embodiment of the system shown in FIG. 1. In the embodiment shown in FIG. 2, the physiology procedure to be performed on the patient is initiated by a manual entry into the ultrasound modality system 16 rather than at the physiology workstation 10. Although the physiology procedure is initiated at the ultrasound modality system 16, it should be understood that the procedure could also be initiated at either the x-ray modality system 14 or any of the other modality systems 18 shown I FIG. 2.

Once the procedure has been initiated at the ultrasound modality system 16, the ultrasound modality system 16 generates a start exam notification message 38 to the physiology workstation 10. Once the physiology workstation 10 receives the start exam notification message, the physiology workstation 10 will obtain the required patient information from the server 12 and will again relay the required start exam notification messages 26 to the modality systems that are required for the physiology procedure being performed on the patient. The start exam notification messages will also be sent back to the modality system where the physiology procedure was initiated by the operator. As with the embodiment shown in FIG. 1, each of the modality systems required for the performance of the physiology procedure will automatically issue a patient info query 30 to the physiology workstation 10, which in turn will reply with the identical patient and exam information such that the physiology workstation 10 and each of the modality systems 14, 16 and 18 will be operating with the identical patient and exam information.

As indicated in the above description, the automatic notification of the beginning of a physiological procedure to each of the modality systems required for the procedure eliminates the requirement that each of the modality systems must be manually initiated for the performance of a physiology procedure. The automatic notification to each of the modality systems from the physiology workstation 10 thus reduces the amount of time required to begin the procedure while also reducing the potential of user data entry errors. The reduction in time and increased accuracy increases the procedural efficiency to start the exam/study, particularly where the exam/study utilizes multiple modality systems. Further, each of the modalities are configured such that once each modality receives the start exam notification message, the modality is auto-triggered to request and exchange the patient information of the current patient undergoing the procedure such that all of the modalities receive the identical patient information. The synchronization between the patient information among the different modalities ensures that the monitoring data received at the physiology workstation 10 can be correctly correlated to a single patient, thus reducing errors associated with prior systems.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A method of integrating a physiology workstation and at least one modality system to conduct a medical procedure on a patient, the method comprising the steps of:
initiating the start of the medical procedure on the physiology workstation;
automatically notifying the at least one modality system of the start of the medical procedure from the physiology workstation;
automatically generating a patient information query from the modality system to obtain patient information; and
transmitting the patient information to the modality system.

2. The method of claim 1 wherein the start of the procedure is manually initiated at the physiology workstation.

3. The method of claim 1 further comprising the steps of:
determining the modality systems required for the procedure; and
automatically notifying all of the required modality systems of the start of the procedure.

4. The method of claim 1 further comprising the step of obtaining the patient information from a storage database upon the initiation of the procedure.

5. The method of claim 1 wherein the modality system is an ultrasound modality system.

6. The method of claim 1 wherein the modality system is an x-ray modality system.

7. The method of claim 1 further comprising the step of transmitting procedure information relating to the procedure being performed on the patient to the modality system upon receipt of the patient information query.

8. A method of integrating the operation of a physiology workstation and a plurality of modality systems to conduct a procedure on a patient, the method comprising the steps of:
initiating a start of a procedure on one of the plurality of modality systems;
automatically notifying the physiology workstation of the start of the procedure;
automatically notifying all of the modality systems of the start of the procedure from the physiology workstation;
automatically generating a patient information query from each of the modality systems to the physiology workstation to obtain patient information and procedure information; and
transmitting the patient information and procedure information to each of the modality systems.

9. The method of claim 8 wherein the procedure is manually initiated at one of the modality systems.

10. The method of claim 8 further comprising the steps of:
determining the modality systems required for the procedure; and
automatically notifying all of the modality systems of the start of the procedure.

11. The method of claim 8 further comprising the step of obtaining the patient information from a storage database upon the initiation of a procedure.

12. The method of claim 8 where at least one of the modality systems is an ultrasound modality system.

13. The method of claim 8 where at least one of the modality systems is an x-ray modality system.

14. A system for conducting a physiology procedure on a patient, the system comprising:
a plurality of modality systems each operable to obtain a patient monitoring data during the physiology procedure; and
a physiology workstation in communication to receive and store a patient monitoring data from the plurality of modality systems during the performance of the physiology procedure, wherein the physiology workstation automatically notifies each of the plurality of modality systems upon an initiation of the procedure; and
wherein each of the plurality of modality systems automatically generate a query to the physiology workstation so as to obtain a patient information upon the initiation of the start of the physiology procedure.

15. The system of claim 14 wherein the plurality of modality systems include at least one of an ultrasound system and an x-ray system.

16. The system of claim 14 wherein the physiology workstation notifies only the modality systems required for the physiology procedure.

17. The system of claim 14 wherein the physiology workstation is a hemodynamic workstation.

18. The system of claim 14 wherein the physiology workstation is an electrophysiology workstation.

19. The system of claim 14 wherein each of the modality systems are configured to automatically notify the physiology workstation when the physiology procedure is initiated in the modality system.
